(19) United States
(12) Patent Application Publication
(10) Pub. No.: US 2011/0053050 A1
(43) Pub. Date: Mar. 3, 2011

(54) METHOD OF FUNCTIONALIZING A CARBON MATERIAL

(75) Inventors: San Hua Lim, Singapore (SG); Chee Kok Poh, Singapore (SG); Jianyi Lin, Singapore (SG)

(73) Assignee: AGENCY FOR SCIENCE, TECHNOLOGY AND RESEARCH, Singapore (SG)

(21) Appl. No.: 12/446,224

(22) PCT Filed: Oct. 18, 2007
(86) PCT No.: PCT/SG2007/000356

§ 371 Date: Nov. 8, 2010

Related U.S. Application Data
(60) Provisional application No. 60/862,014, filed on Oct. 18, 2006.

(51) Int. Cl. H01M 4/92; C07C 51/00; C07C 303/00; C07C 45/00; C07C 41/00; C07C 29/00; C07F 15/00; C07F 13/00; C07F 1/10; C07F 1/12; B82Y 40/00; B82Y 30/00

(52) U.S. Cl. 429/524; 562/498; 562/400; 558/20; 568/445; 568/420; 568/665; 568/579; 568/817; 568/700; 556/136; 556/45; 556/110; 977/847; 977/902

(57) ABSTRACT

The present invention relates to a method of functionalizing a carbon material. A carbon material is contacted with a carboxylic acid, whereby a mixture is formed. The mixture is heated for a suitable period of time at a temperature below the thermal decomposition temperature of the carbon material.

### CROSS-REFERENCE TO RELATED APPLICATIONS

This application makes reference to and claims the benefit of priority of an application for “Functionalization of carbon materials for catalysis applications” filed on Oct. 18, 2006 with the United States Patent and Trademark Office, and there duly assigned Ser. No. 60/862,014. The contents of said application filed on Oct. 18, 2006 is incorporated herein by reference for all purposes, including an incorporation of any element or part of the description, claims or drawings not contained herein and referred to in Rule 20.5(a) of the PCT, pursuant to Rule 4.18 of the PCT.

### FIELD OF THE INVENTION

The present invention relates to a method of functionalizing a carbon material. This functionalization further allows for a method of immobilizing matter, such as particles, on a carbon material.

### BACKGROUND OF THE INVENTION

The chemical functionalization of carbon materials has traditionally posed a challenge in chemistry and engineering. Functionalization enhances solubility and reactivity of carbon materials and provides starting materials for chemical synthesis. The attachment of matter such as particles, e.g. nano-sized metal or metalloid particles, on a carbon support, is also significantly facilitated by the introduction of functional groups on the carbon surface. Furthermore, the discovery of carbon nanostructures such as carbon nanotubes has boosted the interest in carbon materials due to the unique mechanical, electrical and structural properties of such nanostructures.

Investigations have been carried out to find applications of carbon nanotubes in hydrogen storage, electrochemical energy storage, electronic devices and heterogeneous catalysis. A number of studies show that carbon nanotubes can be a better support for Pt catalysts in proton exchange membrane (PEM) fuel cells when compared to traditional carbon black. Matsumoto et al. reported that by using multiwalled carbon nanotubes as a catalyst support in a hydrogen/oxygen fuel cell, a 12 wt % Pt-deposited carbon nanotubes electrode gave 10% higher voltages than 29 wt % Pt-deposited carbon black and reduced the Pt usage by 60% (Matsumoto, T., et al., Chem. Commun. (2004) 7, 840-841). Li et al. demonstrated that Pt catalysts deposited on multi-walled carbon nanotubes had higher activity for direct methanol fuel cell in the high current density region (i.e. at 0.4 V) as compared to that on commercial XC72 carbon black, with 37% higher current density under the same test conditions (Li, W., et al., J. Phys. Chem. B (2003) 107, 26, 6292-6299).

The absence of functional groups renders carbon materials hydrophobic. Carbon nanotubes are even more hydrophobic than other carbon materials due to less defects being present on their surface. It has been shown that the oxidation of carbon nanotubes with HNO₃, KMnO₄, OsO₄, H₂O₂, ozone (O₃), peroxo compounds, azo compounds or diazonium compounds can introduce functional groups such as hydroxyl groups (—OH), carboxyl groups (—COOH), carbonyl groups (—CO), and sulfate groups (—OSO₃H) onto the carbon nanotube surface, thereby for example providing nucleation sites for the deposition of highly dispersed metal particles. International patent application WO 2007/098578 discloses a method of functionalizing carbon nanotubes based on such oxidation processes. In the disclosed method, carbon nanotubes are prior to oxidation dispersed using alkali salts, thereby negatively charging the carbon nanotubes.

Attempts to oxidatively functionalize carbon nanotubes in aqueous ceric sulfate upon exposure to ultrasound have resulted in the destruction of single-walled carbon nanotubes and the conversion of multi-walled carbon nanotubes into graphitic material and amorphous carbon (Luong, J. H. T., et al., Journal of Physical Chemistry B (2005) 109, 4, 1400-1407).

While the currently used surface oxidation methods result in functionalized carbon materials, they are time consuming and often require extensive heating. Dispersed carbon nanotubes with alkali salts have been described to reduce heating and time requirements of the oxidation procedure in some cases, however at the cost of a lengthy dispersion procedure. Current oxidation methods further require filtration and washing in order to remove the oxidant. This would for example increase the cost for commercialization of respective fuel cells operating on the basis of a carbon material.

It is therefore an object of the present invention to provide a method of functionalizing a carbon material that avoids the above described drawbacks or shortcomings of the current techniques.

### SUMMARY OF THE INVENTION

In a first aspect the present invention provides a method of functionalizing a carbon material. The method includes contacting a carbon material with a carboxylic acid. Thereby a mixture is formed. The method further includes heating the mixture for a suitable period of time at a temperature below the thermal decomposition temperature of the carbon material. As a result a functionalized carbon material is formed.

In a second aspect the present invention provides a method of immobilizing matter on a carbon material. The method includes contacting a carbon material with a carboxylic acid. Thereby a mixture is formed. The method further includes heating the mixture for a suitable period of time at a temperature below the thermal decomposition temperature of the carbon material. As a result a functionalized carbon material is formed. The method also includes contacting the functionalized carbon material with a compound capable of forming a covalent bond and/or an ionic bond with the functional groups on the functionalized carbon material.

According to one embodiment of the second aspect particles are formed on the carbon material.

In a third aspect the present invention relates to the use of a carbon material with particles obtained by a method according to the second aspect in catalysis.

### BRIEF DESCRIPTION OF THE DRAWINGS

The invention will be better understood with reference to the detailed description when considered in conjunction with the non-limiting examples and the accompanying drawings.

FIG. 1 shows TEM images of (A) Pt/MWNT (modified using citric acid) at 200 K magnification; (B) Pt/multi-walled carbon nanotubes (modified using citric acid) at 800 K magnification; (C) Pt/XC72 (modified using citric acid) at 200 K magnification; (D) Pt/MWNT (acid refluxed) at 300 K magnification; and (E) Pt/XC-72 at 200 K magnification at 200 K magnification.

FIG. 2 depicts the size distribution of Pt nanoparticles supported on (A) multi-walled carbon nanotubes modified using citric acid, (B) acid refluxed multi-walled carbon nanotubes, (C) on Vulcan carbon black (XC-72) modified using citric acid and (D) the size distribution of Pt nanoparticles supported on as-purchased XC-72.

FIG. 3 depicts ThermoGravimetric (TG) weight loss curves of Pt/multi-walled carbon nanotubes (modified using citric acid) (curve I), Pt/multi-walled carbon nanotubes (acid refluxed), Pt/XC72 (curve III) and Pt/XC72 (modified using citric acid) (curve IV).

FIG. 4 depicts FTIR spectra of (A) multi-walled carbon nanotubes (as-received), multi-walled carbon nanotubes (heated without citric acid), multi-walled carbon nanotubes (acid refluxed) and multi-walled carbon nanotubes (modified using citric acid) respectively, from top to bottom, and (B) XC72 (as purchased) and XC72 (modified using citric acid).

FIG. 5 depicts cyclic voltammograms of Pt/multi-walled carbon nanotubes (modified using citric acid, curve I), Pt/multi-walled carbon nanotubes (acid refluxed, curve II), Pt/XC72 (as purchased, curve III) and Pt/XC72 (modified using citric acid, curve IV), measured at a scan rate of 50 mVs⁻¹ at room temperature in 0.5 M H₂SO₄.

FIG. 6 shows cyclic voltammograms of Pt/multi-walled carbon nanotubes (modified using citric acid, curve I), Pt/multi-walled carbon nanotubes (acid refluxed, curve II), Pt/XC72 (as purchased, curve III) and Pt/XC72 (modified using citric acid, curve IV) measured at a scan rate of 50 mVs⁻¹ at room temperature in 1 M CH₃OH (methanol)+0.5 M H₂SO₄ (sulfuric acid).

FIG. 7 depicts X-ray diffraction patterns of the Pt catalyst supported on [I] multi-walled carbon nanotubes (modified using citric acid), [II] multi-walled carbon nanotubes (acid refluxed), [III] XC72 (as purchased) and [IV] XC72 (modified using citric acid).

### DETAILED DESCRIPTION OF THE INVENTION

The present invention provides a method of functionalizing a carbon material. The method is suitable for any carbon material. In typical embodiments the carbon material is crystalline carbon. The carbon material may for instance include or consist of carbon black, a carbon nanofilament, a buckyball, a 3D carbon sieve, activated carbon, graphite or a carbide-derived carbon material. Illustrative examples of a carbon nanofilament are a carbon nanotube, a carbon nanohorn and a carbon nanowire. Nanotubes are hollow while nanowires are solid. A carbon nanofilament may be of any length and diameter. In some embodiments it may have a diameter of about 1-500 nm, such as about 3-200 nm or about 10-100 nm. Accordingly, the terms “nanofilament” and “nanofiber” as used herein can be used interchangeably. A respective nanotube may have a single wall or multiple walls. A carbon nanotube may also have one or more fullerenes covalently bonded to an outer sidewall thereof, in which case it is generally called a nanobud. A respective carbon material may be metallic, a semiconductor or an insulator. The carbon material may be of any dimension and geometry. In some embodiments these electrically conductive nanofilaments may likewise be carbon nanotubes. In such cases a second plurality of carbon nanotubes is immobilized on the second electrically conductive protrusion. As the plurality of carbon nanotubes immobilized on the first conductive protrusion can be taken to define a plurality of electrically conductive nanofilaments, the present embodiment can also be taken to involve a first plurality of electrically conductive nanofilaments (carbon nanotubes) on a first conductive protrusion and a second plurality of electrically conductive nanofilaments on a second conductive protrusion.

Where one or more carbon nanotubes are used as the carbon material, they may be pre-formed according to any desired method (see e.g. Rao, C. N. R., et al., Chem Phys Chem [2001] 2, 78-105, included herein by reference in its entirety). A carbon nanotube is a cylinder of rolled up graphitic sheets. Both single- and multi-walled carbon nanotubes are known and can equally be used in the method of the present invention. The carbon nanotubes may be of any desired length, such as in the range from about 10 nm to about 10 μm. The conductivity of the carbon nanotubes used may be freely selected according to any specific requirements of particular embodiments. Depending on the arrangement of the carbon hexagon rings along the surface of the nanotubes, carbon nanotubes can be metallic or semiconducting. Any such carbon nanotubes may be used in a method according to the present invention.

The carbon material used (as a starting material) in the present invention may be without any functional groups or have some or many functional groups of any desired type. Typically the method of the invention will be used on carbon material that is at least essentially without functional groups or poorly functionalized, because the need to use the method of the invention will usually be the highest for such starting material. The term “functionalizing” generally refers to the introduction of functional groups to the carbon material. Any functional group may be introduced into the carbon material. Typical functional groups introduced in the course of the method of the invention include, but are not limited to, —COOH (carboxy), —CHO (aldehyde), —CO— (carbonyl), —OSO₃H (sulfate), —OSO— (sulfonyl), —O— (oxo) and —OH (hydroxy). Other functional groups, which may already be present in the carbon material, or which in some embodiments be generated during the method of the invention, include for example —NH₂ (amino), —NO (nitro), —Br (bromo), —Cl (chloro) and —F (fluoro). It may be desired to consider the intended use of the functionalized material obtained by the method of the present invention when selecting the starting material. The presence of certain functional groups in the carbon starting material may in some embodiments be disadvantageous for an intended subsequent use. As an illustrative example, some functional groups such as a —Cl group, may in some cases act as a poison for a metal catalyst, and may thus affect (including derogate) a desired use of a carbon material obtained by a method according to the invention as a catalyst. Those skilled in the art will be aware that the same caution should be taken when selecting a carbon (starting) material with regard to the content of e.g. sulfur of the same.

The carbon (starting) material may be provided in any form, such as in form of a powder, an aerogel (e.g. of carbon nanotubes [for an indication on the handling of a respective aerogel see e.g. Bryning, M. B., et al., Advanced Materials (2007) 19, 661-664]), one or more solid blocks, a suspension, a dispersion or a solution. Where a solution, suspension or dispersion is provided, a liquid such as a commercially available solvent or water is used. Any desired liquid can be employed, whether an aqueous or non aqueous liquid, an organic liquid (solvent), or a nonpolar aprotic, nonpolar protic, dipolar protic, dipolar aprotic, or an ionic liquid. Examples of nonpolar aprotic liquids include, but are not limited to, hexane, heptane, cyclo-hexane, benzene, toluene, pyridine, dichloromethane, chloroform, carbon tetrachloride, carbon disulfide, tetrahydrofuran, dioxane, diethyl ether, diisopropylether, ethylene glycol monobutyl ether or tetrahydrofuran. Examples of dipolar aprotic liquids are methyl ethyl ketone, methyl isobutyl ketone, acetone, cyclohexanone, ethyl acetate, isobutyl isobutyrate, ethylene glycol diacetate, dimethylformamide, acetonitrile, N,N-dimethyl acetamide, nitromethane, acetonitrile, N-methylpyrrolidone, and dimethylsulfoxide. Examples of polar protic liquids are water, methanol, ethanol, butyl alcohol, formic acid, dimethylarsinic acid [(CH₃)₂AsO(OH)], N,N-dimethyl-formamide, N,N-diisopropylethylamine, or chlorophenol. Examples of nonpolar protic liquids are acetic acid, tert.-butyl alcohol, phenol, cyclohexanol, or aniline. Two illustrative examples of ionic liquids are 1,3-dialkylimidazolium-tetrafluoroborates and 1,3-dialkylimidazolium-hexafluoroborates.

In some embodiments the liquid is a polar ionic liquid. Examples of a polar ionic liquid include, but are not limited to, 1-ethyl-3-methylimidazolium tetrafluoroborate, N-butyl-4-methylpyridinium tetrafluoroborate, 1,3-dialkylimidazolium-tetrafluoroborate, 1,3-dialkylimidazolium-hexafluoroborate, 1-ethyl-3-methylimidazolium bis(pentafluoroethyl)phosphinate, 1-butyl-3-methylimidazolium tetrakis(3,5-bis(trifluoromethylphenyl)borate, tetrabutylammonium bis(trifluoromethyl)imide, ethyl-3-methylimidazolium trifluoromethanesulfonate, 1-butyl-3-methylimidazolium methylsulfate, 1-n-butyl-3-methylimidazolium ([bmim]) octylsulfate, and 1-n-butyl-3-methylimidazolium tetrafluoroborate. Examples of a non-polar liquid include, but are not limited to mineral oil, hexane, heptane, cyclohexane, benzene, toluene, di-chloromethane, chloroform, carbon tetrachloride, carbon disulfide, dioxane, diethyl ether, diisopropylether, methyl propyl ketone, methyl isoamyl ketone, methyl isobutyl ketone, cyclohexanone, isobutyl isobutyrate, ethylene glycol diacetate, and a non-polar ionic liquid. Examples of a non-polar ionic liquid include, but are not limited to, 1-ethyl-3-methylimidazolium bis-[(trifluoromethyl)sulfonyl]amide bis(triflyl)amide, 1-ethyl-3-methylimidazolium bis[(trifluoromethyl)sulfonyl]amide trifluoroacetate, 1-butyl-3-methylimidazolium hexafluorophosphate, 1-hexyl-3-methylimidazolium bis(trifluoromethylsulfonyl)imide, 1-butyl-3-methylimidazolium bis(trifluoromethylsulfonyl)imide, trihexyl(tetradecyl)phosphonium bis[oxalato(2-)]borate, 1-hexyl-3-methyl imidazolium tris(pentafluoroethyl)trifluorophosphate, 1′-butyl-3-methyl-imidazolium hexafluorophosphate, tris(pentafluoroethyl)trifluorophosphate, trihexyl(tetradecyl)phosphonium, N″-ethyl-N,N,N′,N′-tetramethylguanidinium, 1-butyl-1-methylpyrrolidinium tris(pentafluoroethyl)trifluorophosphate, 1-butyl-1-methylpyrrolidinium bis(trifluoromethylsulfonyl)imide, 1-butyl-3-methyl imidazolium hexafluorophosphate, 1-ethyl-3-methylimidazolium bis(trifluoromethylsulfonyl)imide and 1-n-butyl-3-methylimidazolium.

As an illustrative example, single-walled carbon nanotubes may be provided as a dispersion in an aromatic organic polymer such as poly(9,9-doctylfluorenyl-2,7-diyl) as described by Nish et al. (Nature Nanotech. (2007) 2, 10, 640-646).

In a method according to the invention the carbon (starting) material is contacted with a carboxylic acid. Any carboxylic acid, typically an organic carbocylic acid, may be used. The carboxylic acid may be of any desired (molecular) length and include any desired number of heteroatoms and functional groups. Examples of a respective functional group include, but are not limited to, a halogen, a hydroxyl-, a thiol-, a dithiane-, a seleno-, a carboxyl-, carbonyl-, amino-, imino-, amido-, imido-, azido-, diazo-, cyano-, isocyano-, thiocyano-, nitro-, nitroso-, sulfo-, sulfido-, sulfonyl- (e.g. a trifluoromethyl sulfonyl-, p-toluenesulfonyl, bromobenzene-sulfonyl, nitrobenzenesulfonyl-, or a methane-sulfonyl), silyl-, silano- or a siloxy-group. In some embodiments the (organic) carboxylic acid is an aliphatic, a cycloaliphatic, an aromatic, an arylaliphatic, or an arylcycloaliphatic carboxylic acid with a main chain of a length of 2 to about 20 carbon atoms, such as about 3 to about 20 carbon atoms, about 3 to about 15 carbon atoms or about 3 to about 10 carbon atoms. In addition the main chain may in some embodiments include 0 to about 5 heteroatoms, such as about 1, about 2, about 3, about 4 or about 5 heteroatoms. Examples of suitable heteroatoms include, but are not limited to, N, O, S, Se and Si.

The term “aliphatic” means, unless otherwise stated, a straight or branched hydro-carbon chain, which may be saturated or mono- or poly-unsaturated and include heteroatoms (see above). An unsaturated aliphatic group contains one or more double and/or triple bonds (alkenyl or alkinyl moieties). The branches of the hydrocarbon chain may include linear chains as well as non-aromatic cyclic elements. The hydrocarbon chain, which may, unless otherwise stated, be of any length, and contain any number of branches. Typically, the hydrocarbon (main) chain includes 1 to about 5, to about 10, to about 15 or to about 20 carbon atoms. Examples of alkenyl radicals are straight-chain or branched hydrocarbon radicals which contain one or more double bonds. Alkenyl radicals normally contain about two to about twenty carbon atoms and one or more, for instance two, double bonds, such as about two to about ten carbon atoms, and one double bond. Alkynyl radicals generally contain about two to about twenty carbon atoms and one or more, for example two, triple bonds, such as about two to about ten carbon atoms, and one triple bond. Examples of alkynyl radicals are straight-chain or branched hydrocarbon radicals which contain one or more triple bonds. Examples of alkyl groups are methyl, ethyl, propyl, butyl, pentyl, hexyl, heptyl, octyl, nonyl, decyl, the n isomers of these radicals, isopropyl, isobutyl, isopentyl, sec-butyl, tent-butyl, neopentyl, 3,3-dimethylbutyl. Both the main chain as well as the branches may furthermore contain heteroatoms as for instance N, O, S, Se or Si or carbon atoms may be replaced by these heteroatoms.

The term “alicyclic” means, unless otherwise stated, a non-aromatic cyclic moiety (e.g. hydrocarbon moiety), which may be saturated or mono- or poly-unsaturated. The cyclic hydrocarbon moiety may also include fused cyclic ring systems such as decalin and may also be substituted with non-aromatic cyclic as well as chain elements. The main chain of the cyclic hydrocarbon moiety may, unless otherwise stated, be of any length and contain any number of non-aromatic cyclic and chain elements. Typically, the hydrocarbon (main) chain includes 3, 4, 5, 6, 7 or 8 main chain atoms in one cycle. Examples of such moieties include, but are not limited to, cyclopentyl, cyclohexyl, cycloheptyl, or cyclooctyl. Both the cyclic hydrocarbon moiety and, if present, any cyclic and chain substituents may furthermore contain heteroatoms, as for instance N, O, S, Se or Si, or a carbon atom may be replaced by these heteroatoms. The term “alicyclic” also includes cycloalkenyl moieties that are unsaturated cyclic hydrocarbons, which generally contain about three to about eight ring carbon atoms, for example five or six ring carbon atoms. Cycloalkenyl radicals typically have a double bond in the respective ring system. Cycloalkenyl radicals may in turn be substituted.

The term “aromatic” means a planar cyclic hydrocarbon moiety of conjugated double bonds, which may be a single ring or include multiple fused or covalently linked rings, for example, 2, 3 or 4 fused rings. The term aromatic also includes alkylaryl. Typically, the hydro-carbon (main) chain includes about 5, 6, 7 or about 8 main chain atoms in one cycle. Examples of such moieties include, but are not limited to, cylcopentadienyl, phenyl, napthalenyl-, [10]annulenyl-(1,3,5,7,9-cyclodecapentaenyl-), [12]annulenyl-, [8]annulenyl-, phenalene (perinaphthene), 1,9-dihydropyrene, chrysene (1,2-benzophenanthrene). An example of an alkylaryl moiety is benzyl. The main chain of the cyclic hydrocarbon moiety may, unless otherwise stated, be of any length and contain any number of heteroatoms, as for instance N, O and S. Examples of such heteroaromatic moeities (which are known to the person skilled in the art) include, but are not limited to, furanyl-, thiophenyl-, naphtyl-, naphthofuranyl-, anthrathiophenyl-, pyridinyl-, pyrrolyl-, quinolinyl, naphthoquinolinyl-, quinoxalinyl-, indolyl-, benzindolyl-, imidazolyl-, oxazolyl-, oxoninyl-, oxepinyl-, benzoxepinyl-, azepinyl-, thiepinyl-, selenepinyl-, thioninyl-, azecinyl- (azacyclodecapentaenyl-), diazecinyl-, azacyclododeca-1,3,5,7,9,11-hexaene-5,9-diyl-, azozinyl-, diazocinyl-, benzazocinyl-, azecinyl-, azaundecinyl-, thia[11]annulenyl-, oxacyclotrideca-2,4,6,8,10,12-hexaenyl- or triazaanthracenyl-moieties.

By the term “arylaliphatic” is meant a hydrocarbon moiety, in which one or more aromatic moieties are substituted with one or more aliphatic groups. Thus the term “arylaliphatic” also includes hydrocarbon moieties, in which two or more aryl groups are connected via one or more aliphatic chain or chains of any length, for instance a methylene group. Typically, the hydrocarbon (main) chain includes 5, 6, 7 or 8 main chain atoms in each ring of the aromatic moiety. Examples of arylaliphatic moieties include, but are not limited, to 1-ethyl-naphthalene, 1,1′-methylenebis-benzene, 9-isopropylanthracene, 1,2,3-trimethyl-benzene, 4-phenyl-2-buten-1-ol, 7-chloro-3-(1-methylethyl)-quinoline, 3-heptyl-furan, 6-[2-(2,5-diethyl-phenyl)ethyl]-4-ethyl-quinazoline or, 7,8-dibutyl-5,6-diethyl-isoquinoline.

As already indicated above, each of the terms “aliphatic”, “alicyclic”, “aromatic” and “arylaliphatic” as used herein is meant to include both substituted and unsubstituted forms of the respective moiety. Substituents may be any functional group (see above for examples).

In some embodiments the carboxylic acid is a hydroxy carboxylic acid, a dicarboxylic acid (including a tricarboxylic acid), an amino acid or any mixture thereof. To provide a number of illustrative examples, the organic carboxylic acid may be oxalic acid, ascorbic acid, citric acid, glycolic acid, tartaric acid, malic acid, maleic acid, adipic acid, lactic acid, salicylic acid or any mixture or other combination thereof. Examples of a suitable amino acid include, but are not limited to, glutamine, lysine, histidine, serine, threonine, tyrosine, cystine, cysteine, arginine, proline, glutamic acid, aspartic acid, asparagine, glutamine or any mixture thereof. The carboxylic acid may be solid or liquid and it may also be provided in form of a solution or dispersion. Any liquid may be used in this regard (see above). Accordingly, the carbon material and the carboxylic acid may be contacted in solid form or one of them may be provided in a liquid. The carbon material may for instance be contacted with a solution of the carboxylic acid or the carboxylic acid may be contacted with the carbon material by adding a solution of the carboxylic acid to the carbon material. In some embodiments the carbon material, the carboxylic acid or both may dissolve, precipitate, form a suspension, a gel, a dispersion or any combination thereof, upon contacting the two. Contacting the carbon material with the carboxylic acid may for instance include forming a suspension and/or a solution of the carboxylic acid and the carbon material in a solvent.

In a method according to the present invention a mixture of the carbon material and the carboxylic acid is formed. A respective mixture may be intermixed, e.g. blended, stirred, etc. In some embodiments the mixture of the carbon material and the carboxylic acid is dried, which may be done for any desired period of time. Drying the mixture may for example include applying reduced pressure, applying a stream of a gas, elevated temperature or irradiation such as exposure to microwaves. Drying may also be carried out by exposing the mixture to the atmosphere at room temperature. In some embodiments drying may include applying heat (in air, under reduced pressure, under stream of a gas etc.), including heating with a heat gun. In embodiments where heat is applied, it may be desired to heat the mixture only at, or up to, a temperature below the thermal decomposition temperature of the carboxylic acid in order to avoid derogation or decomposition of the same. As an illustrative example, where citric acid is used as the carboxylic acid, it may be desired to select a heating temperature that is below 175° C., which is the thermal decomposition temperature of citric acid. Drying the mixture may be desired in embodiments where the concentration of the carboxylic acid and the homogeneity of the same, or of the entire mixture, is to be increased.

During drying the mixture of the carbon material and the carboxylic acid, as explained above, the surface of the carbon material generally remains at least essentially unchanged. Functionalization of the respective surface does typically not occur during this drying process. Accordingly drying may be carried out to remove (typically evaporate) any solvent or other undesired liquid without further, at least essentially, affecting the carbon material and the carboxylic acid. Drying the mixture of the carbon material and the carboxylic acid may result in the formation of a paste. Such a paste will typically include the carbon material and the carboxylic acid in the mixture. A respective paste may be of any consistency.

Citric acid, as well as inter alia hydroxyquinoline and 3-methyl-1-phenyl-pyrazolone-5, has so far only been found to improve the adsorption of heavy metal ions onto carbon material (Chen, J. P., et al., Carbon (2003) 41, 1979-1986) after being adsorbed thereto itself. It has been speculated that this modification were assisted by an effect of citric acid being particularly well adsorbed to a carbon surface. The method of the present invention is as good as simple, however at the same time it provides functionalized carbon materials.

In a method according to the invention the mixture of the carbon material and the carboxylic acid, which may have been dried (including a paste), is heated at a temperature below the thermal decomposition temperature of the carbon material. The thermal decomposition temperature may be the ignition point, e.g. the flash point or the fire point of the carbon material. The term “ignition point” includes the term “flash point” and the term “fire point” in relation to liquids and solid materials. The flash point is the lowest temperature at which vapour of a liquid can form an ignitable mixture in air near the surface of the liquid. Below this temperature insufficient vapour of the carbon material is available to allow for combustion to occur. The fire point is the temperature at which the flame becomes self-sustained so as to continue burning the carbon material. The fire point is usually a few degrees above the flash point. As an illustrative example, where activated carbon is used, the flashpoint thereof may be as low as about 260° C., depending on the source of the carbon material. For carbon black the flashpoint may be about 325° C., likewise depending on the source of the carbon material. Graphite may start burning at temperatures around 650° C. Multiwalled carbon nanotubes may start burning at about 500° C. while single walled carbon nanotubes may do so at about 650° C. For a particular carbon material the upper temperature suitable in the method of the invention, such as the flashpoint, die-down or decomposition temperature, may easily be determined experimentally where required.

Heating the mixture of the carbon material and the carboxylic acid may be carried out under, or in the presence of, any gas. The heating can conveniently be carried out in/under air. The use of an inert gas atmosphere such as nitrogen or argon may in some embodiments be desired in order to preserve functional groups generated from degradation by the applied elevated temperature. A respective gas may also be exchanged during heating of the mixture. Air may, for instance, be present during an initial heating phase and then gradually, rapidly or at once be replaced by an inert gas to any extent.

The mixture of the carbon material and the carboxylic acid is heated for a suitable period of time for the formation of a functionalized carbon material. The exact time range suitable for a selected combination of a carboxylic acid and a carbon material may easily be determined by a series of tests. Generally a certain minimum time period is required in order to allow for the formation of functional groups. If it is desired to at least essentially or to entirely remove any carboxylic acid during the heating of the mixture, the respective time period to achieve this removal may be longer than the minimum time interval, after which functional groups are formed. Furthermore exposure of functional groups to heat will result in their degradation. Therefore at a certain time interval of heating removal of functional groups and generation of new functional groups will be at equilibrium. At prolonged time intervals—also depending on the materials and temperatures used—removal of functional groups may be the predominant process. As a general orientation, in some embodiments heating the mixture of the carbon material and the carboxylic acid at a temperature below the thermal decomposition temperature (e.g. the ignition point) of the carbon material is carried out for about 2 or about 3 hours (such as about 1 hour) or less, such as for instance about 10 min, about 20 min, about 30 min, about 40 min or about 50 min. The mixture of the carbon material and the carboxylic acid may in some embodiments be heated for a time interval in the range from about 15 minutes to about 1.5 hours, such as about 20 minutes to about 1 hour or about 30 minutes to about 1 hour. As an illustrative example, functionalizing carbon nanotubes with citric acid at temperatures above 175° C. (the thermal decomposition temperature of citric acid) has been observed to often be incomplete at time intervals below around 30 minutes. If the reaction time exceeds about 1 hour, a reduction in the number of functional groups can be observed when using citric acid as carboxylic acid for functionalization of carbon nanotubes. Some functional groups might for example be destructed due to reactions with oxygen in air. However, such conditions that may lead to incomplete functionalization are of course still encompassed in the present invention since they are still able to provide the desired modification.

As already mentioned above, the mixture of a carbon material and a carboxylic acid may in some embodiments be heated at a temperature above the thermal decomposition temperature of the carboxylic acid. In such embodiments the carboxylic acid is at least essentially removed during the functionalization process, thereby redundantizing subsequent purification steps in this regard. Heating the mixture of a carbon material and a carboxylic acid (which may be dried, supra) at a temperature below the flash point of the carbon material may be carried out by any means (see above for examples). In some embodiments the mixture is exposed to a hot gas. In some embodiments the heating is performed in a space, such as a chamber, designed for applying heat to matter. The heating may for instance be carried out in a furnace.

The functionalized carbon material may include any functional group. Particularly in embodiments where the heating has been carried out in an atmosphere that includes oxygen, oxygen containing functional groups such as —COOH, —CHO, —CO—, —OSO₃H, —OSO₂H, —SO₃R, —OSOR, —NO₂ (nitro), —NO (nitroso) or —OH may be present in the functionalized carbon material (see also above). The letter “R” represents any aliphatic, cycloaliphatic, aromatic, arylaliphatic or arylcycloaliphatic group (see above). Other functional groups such as NH₂, Br, Cl and F may be generated or may have been present in the carbon starting material (i.e., before undergoing the method of the invention). Functional groups on the surface may be further modified, for example to obtain more reactive functional groups. As an illustrative example, thionyl chloride, SOCl₂, may be used to convert carboxyl groups on a carbon material into carboxylic acid chloride groups as described by Rios et al. (Materials Research (2003) 6, 2, 129-135).

Acid treatment of carbon nanotubes is known to involve a cutting thereof, in particular of single walled carbon nanotubes, resulting in a breakdown of a carbon nanotube network (see e.g. Dumitrescu, I., et al., J. Phys. Chem. (2007) 111, 12944-12953). The method of the present invention is expected to be milder than current oxidative processes and involve less cutting since the method of the invention involves the use of a carboxylic acid, which is a weaker acid than for example HNO₃. It is also recalled in this regard that the method of the present invention requires an operation time that is typically relatively short and a temperature that is typically relatively low when compared to methods currently used in the art. Functionalized carbon nanotubes obtained by the method of the invention may accordingly be included in a nanotube network. Upon selection of an appropriate carboxylic acid, temperature and time interval, there ought to be conditions identifiable, under which such a nanotube network is, at least essentially, preserved.

In some embodiments the functionalized carbon material may be contacted with a compound capable of forming a covalent bond, including a coordinative bond, with the functional groups on the functionalized carbon material. In some embodiments the functionalized carbon material may be contacted with a compound capable of forming an ionic bond with the respective functional groups on its surface. A respective compound used may also be able to form both covalent and ionic bonds with functional groups on the surface of the carbon material, for instance via different moieties of the molecule of the respective compound. In this regard the invention also provides a method of immobilizing matter on the carbon material. In some embodiments an anchor may be formed on the carbon material by a reaction with a compound capable of forming a covalent bond or ionic bond with the respective functional groups. A respective compound may be hydrocarbon-based (including polymeric) and include nitrogen-, phosphorus-, sulphur-, carbon-, halogen- or pseudohalogen groups. Illustrative examples include, but are not limited to, an amino group, an aldehyde group, a thiol group, a carboxy group, an ester, an anhydride, a sulphonate, a sulphonate ester, an imido ester, a silyl halide, an epoxide, an aziridine, a phosphoramidite and a diazoalkane. An illustrative example of a respective anchor-forming compound is toluene 2,4-diisocyanate. This anchor-forming compound can then for instance be used to carry out an anionic ring-opening polymerization of s-caprolactam (Yang, M., et al., Carbon (2007) 45, 2327-2333).

In some embodiments the anchor-forming compound may be a receptor molecule for a target molecule such as a protein, a nucleic acid, a polysaccharide or any combination thereof. In such embodiments the anchor-forming compound and such a target molecule may define a specific binding pair. Examples of a respective receptor molecule include, but are not limited to, an immunoglobulin, a fragment thereof, a mutein based on a polypeptide of the lipocalin family, a glubody, a domain antibody (a diabody, a triabody or a decabody), a protein based on the ankyrin or crystalline scaffold, an avimer, an AdNectin, a tetranectin, the T7 epitope, maltose binding protein, the HSV epitope of herpes simplex virus glycoprotein D, the hemagglutinin epitope, and the myc epitope of the transcription factor c-myc, an oligo-nucleotide, an oligosaccharide, an oligopeptide, biotin, dinitrophenol, digoxigenin and a metal chelator (cf. also below). As an illustrative example, a respective metal chelator, such as ethyl-lenediamine, ethylenediaminetetraacetic acid (EDTA), ethylene glycol tetraacetic acid (EGTA), diethylenetriaminepentaacetic acid (DTPA), N,N-bis(carboxymethyl)glycine (also called nitrilotriacetic acid, NTA), 1,2-bis(o-aminophenoxy)ethane-N,N,N′,N′-tetraacetic acid (BAPTA), 2,3-dimercapto-1-propanol (dimercaprol), porphine or heme may be used in cases where the target molecule is a metal ion. As an example, EDTA forms a complex with most monovalent, divalent, trivalent and tetravalent metal ions, such as e.g. silver (Ag⁺), calcium (Ca²⁺), manganese (Mn²⁺), copper (Cu²⁺), iron (Fe²⁺), cobalt (Co³⁺) and zirconium (Zr⁴⁺), while BAPTA is specific for Ca²⁺. In some embodiments a respective metal chelator in a complex with a respective metal ion or metal ions defines the linking moiety. Such a complex is for example a receptor molecule for a peptide of a defined sequence, which may also be included in a protein. As an illustrative example, a standard method used in the art is the formation of a complex between an oligohistidine tag and copper (Cu²⁺), nickel (Ni²⁺), cobalt (Co²⁺), or zinc (Zn²⁺) ions, which are presented by means of the chelator nitrilotriacetic acid (NTA).

In some embodiments the compound capable of forming a covalent bond or an ionic bond with the functional groups on the functionalized carbon material may be polymerisable. In such embodiments the method of the present invention may for example be used to form a carbon material (such as carbon nanotubes) that is polymer-grafted (cf. Liu, M., et al., J. Phys. Chem. C (2007) 111, 2379-2385; Gao, C., et al., J. Phys. Chem. B (2005) 109, 11925-11932; Yang et al., 2007, supra). In some embodiments the compound capable of forming a covalent bond or an ionic bond with the functional groups on the functionalized carbon material is an oligomer or a polymer.

In some embodiments the compound capable of forming a covalent bond and/or an ionic bond with the functional groups on the functionalized carbon material used may be a metal compound (for example, a transition metal or a noble metal) or a metalloid compound. Examples of suitable metalloids include, but are not limited to silicon, boron, germanium, antimony and composites thereof. Examples of suitable metals include, but are not limited to iron (e.g. steel), aluminum, gold, silver, platinum, palladium, rhodium, zirconium, chromium, ruthenium, rhenium, nickel, cobalt, tin, copper, titanium, zinc, aluminum, lead and composites (including alloys) thereof. Upon contacting the functionalized carbon material with a respective metal or metalloid compound a covalent bond (including a coordinative bond) or an ionic bond may be formed. In some embodiments thereby one or more metal or metalloid particles are forming on the surface of the carbon material. As an illustrative example, the addition of an aqueous solution of platinum chloride (PtCl₂) to functionalized carbon nanotubes has previously been shown to result in the formation of platinum nanoclusters thereon (Yu, R., et al., Chem. Mater. (1998) 10, 718-722). Accordingly the invention also provides a method of forming one or more particles on a carbon material. The respective particle may be a metal particle, a metalloid particle, a metal oxide particle, a metalloid oxide particle or include any mixture of a metal, a metalloid, a metal oxide or a metalloid oxide.

The compound that is capable of forming a covalent bond and/or an ionic bond with the functional groups on the functionalized carbon material may be provided in any form. In some embodiments it is provided in a solvent. In some embodiments the compound capable of forming a covalent bond or an ionic bond with the respective functional groups is included in a particle, including being present on the surface thereof. Upon contacting the functionalized carbon material with such a functionalized particle the particle is immobilized on the carbon material.

In some embodiments the particle formed on the carbon material is a nanoparticle. It may in some embodiments have a diameter of less than about 500 nm, such as less than about 100 nm, less than about 50 nm, less than about 30 nm or less than about 15 nm. Such a particle may include portions, such as a core of matter different from the remaining particle. As an illustrative example, the particle may for instance have a metal core with a metal oxide shell. In some embodiments alloy nanoparticles are formed, which may for example include two or more transition metals.

A method according to the invention may also include comminuting the functionalized carbon material. It may for example be grinded, exposed to shredding or pounded. In some embodiments the functionalized carbon material is comminuted before contacting the same with a metal compound capable of forming a covalent bond or an ionic bond with the functional groups on the functionalized carbon material. In other embodiments the functionalized carbon material carrying an anchor-forming compound, a metal or metalloid compound, a particle or other matter is comminuted.

A carbon material with particles obtained by a method according to the present invention may be used in catalysis. In some embodiments the catalysis is in oxidation and/or in reduction in a fuel cell (cf. Matsumoto et al., 2004, supra). As an illustrative example, the carbon material may serve as a catalyst support for catalyst materials/particles such as palladium or platinum (including PtSnO₂ or PtRu) particles located thereon. Platinum is for instance highly catalytic in hydrocarbon or hydrogen oxidation. Platinum is however a costly metal. Providing platinum nanoparticles on a carbon support inter alia maximizes the platinum surface thereby significantly reducing catalyst amounts and thus catalyst costs.

Likewise, a carbon material with particles immobilized thereon such as Pt/C is effective in reduction in a fuel cell. As an example, it may be used as the cathode catalyst in H₂—O₂ Proton Exchange Membrane (PEM) fuel cells or direct methanol fuel cells. Pt or PtRu catalyze the reaction: O₂+4H⁺+4e→H₂O. As further illustrative examples, Pt/C, Re/C and PtRe/C have also been used for glycerol conversion to syngas, being better catalysts than those supported on oxides (Simonetti, D. A., et al., J. Catal. (2007) 247, 2, 298-306; Soares, P. R., et al., Angew. Chem. Int. Ed. (2006) 45, 24, 3982-3985).

A respective fuel cell may be of any type, for instance a proton exchange membrane fuel cell or a direct methanol fuel cell. Carbon material with particles obtained by the method of the invention may for instance be included in, or form an electrode. Electrodes of carbon nanotubes with immobilized platinum particles as well as carbon black with immobilized platinum particles have previously been shown to be effective in electrolyte fuel cells (supra). Electrodes of carbon nanotubes with immobilized platinum particles have previously been shown to have a several fold higher performance than respective electrodes of carbon black with immobilized platinum particles (e.g. Matsumoto et al., 2004, supra). Techniques of characterizing immobilized nanoparticles in a fuel cell are well known in the art (see e.g. Liu, Z., et al., Materials Chemistry & Physics (2007) 105, 2-3, 222-228).

In order that the invention may be readily understood and put into practical effect, particular embodiments will now be described by way of the following non-limiting examples.

### Exemplary Embodiments of the Invention

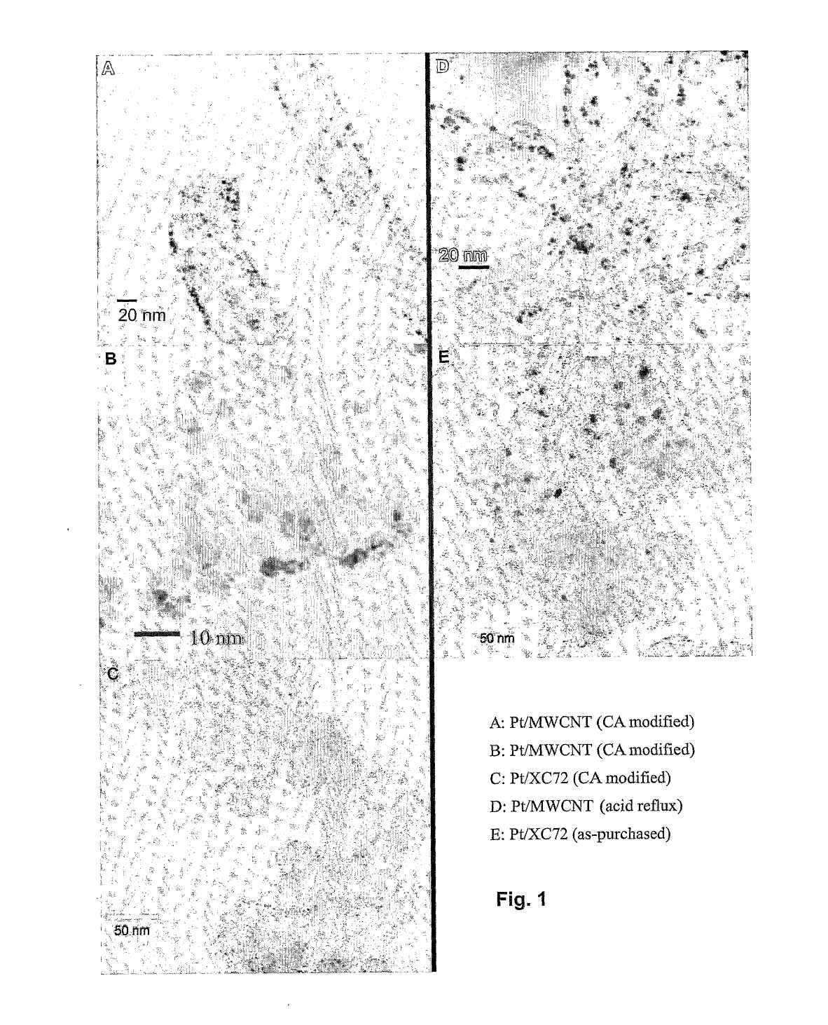

FIG. 1 depicts transmission electron microscopy (TEM) images of platinum nanoparticles supported on different carbon materials. In FIG. 1A and FIG. 1B, Pt nanoparticles supported on citric acid modified carbon nanotubes are seen to be highly dispersed with much better dispersion than those on acid refluxed multi-walled carbon nanotubes (FIG. 1D) and on XC-72 (FIG. 1E). Pt nanoparticles supported on citric acid modified XC-72 (FIG. 1C) showed an excellent dispersion. MWCNT=multiwalled carbon nanotubes, CA modified=modified by means of the method of the invention, using citric acid as the carboxylic acid; Pt=platinum.

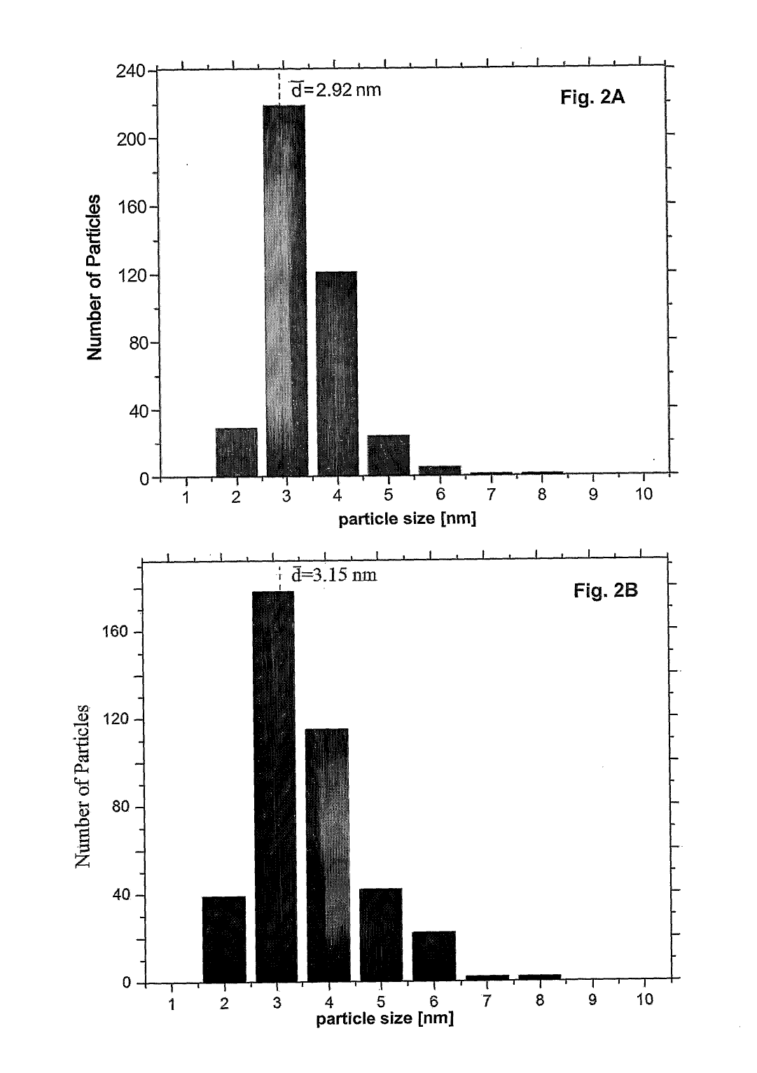

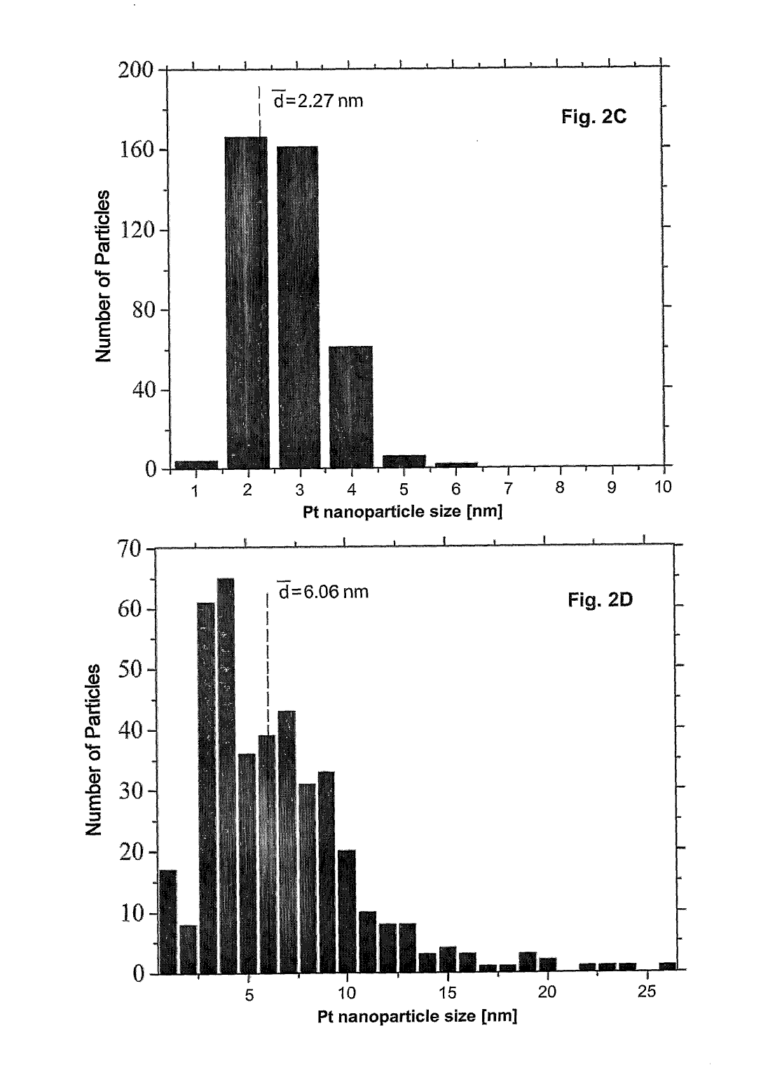

As reported previously (Yu, W., et al., Langmuir (1999) 15, 6; Chen, W. X.; et al., Chem. Commun. (2002) 2588; Liu, Z., et al., J. Mater. Chem. (2003) 13, 3049), the microwave-synthesized Pt nanoparticles have a narrow particle size distribution. The histograms in FIG. 2 give the mean particle size of the Pt nanoparticles, being approximately 2.92±0.77, 3.15±1.02 and 2.27±1.73 nm for Pt/carbon nanotubes (modified using citric acid) (FIG. 2A), Pt/carbon nanotubes (acid refluxed) (FIG. 2B) and Pt/XC-72 (modified using citric acid) (FIG. 2C) respectively. The mean particle size of the Pt nanoparticles was 6.1±4.0 nm for Pt/XC72 (as-purchased, FIG. 2D). The density of Pt particle numbers on the carbon supports, estimated from the TEM images, is around 3.3×10¹⁶/m², 1.3×10¹⁶/m², 5.43×10¹⁶/m² and 1.94×10¹⁶/m² for Pt/multi-walled carbon nanotubes (modified using citric acid), Pt/multi-walled carbon nanotubes (acid refluxed), Pt/XC72 (modified using citric acid) and Pt/XC72. Under identical preparation procedures, the high Pt particle number per unit area and small particle sizes are significantly important in fuel cell application since it may increase Pt utilization and reduce limitation of mass transport and ohmic resistance (Srinivasan, S., et al., J. Power Sources (1990) 29, 3-4, 367-387; Shao, Z., et al., J. Power Sources (1999) 79, 1, 82-85). The poorer dispersion of Pt nanoparticles on carbon black might be due to relatively lower concentration of functional groups on the respective surface. Most Pt nanoparticles on carbon black may be spontaneously deposited on surface defects, while the homogeneous dispersion of Pt nanoparticles on the carbon nanotubes is attributed to the functional groups that are distributed on the surface of carbon nanotubes (Guo, D. J., & Li, H. L., Electroanal. (2005) 17, 10, 869-872; Zoval, J. V., et al., J. Phys. Chem. B (1998) 102, 7, 1166-1175). The functionalization of carbon materials is effective using citric acid as a carboxylic acid in the method of the invention, since the surface density of Pt nanoparticles is higher on both citric acid modified multi-walled carbon nanotubes and XC72.

Thermogravimetric analysis (TGA) weight loss curves of Pt/multi-walled carbon nanotubes (modified using citric acid), Pt/multi-walled carbon nanotubes (acid refluxed), Pt/XC72 and Pt/XC72 (modified using citric acid) upon heating in oxygen with increasing temperature are shown in FIG. 3. The carbon supports of Pt/multi-walled carbon nanotubes (modified using citric acid), Pt/multi-walled carbon nanotubes (acid refluxed), Pt/XC72 and Pt/XC72 (modified using citric acid) were completely burned at 650 (curve I), 625 (curve II), 560 (curve III) and 511 (curve IV)° C. respectively. The Pt loading of the catalyst is estimated to be 15.4 wt % on citric acid modified multi-walled carbon nanotubes as compared to 12.6 wt % on acid refluxed multi-walled carbon nanotubes, 13.0 wt % on XC72 and 14.6 wt % on citric acid modified XC72. The fact that Pt/multi-walled carbon nanotubes (modified using citric acid) and Pt/XC72 (modified using citric acid) have higher loading but smaller Pt nanoparticles implies that the method of the invention creates more functional groups on the surface of carbon materials, and thus more Pt nanoparticles are formed with the surface functional groups as nucleation sites.

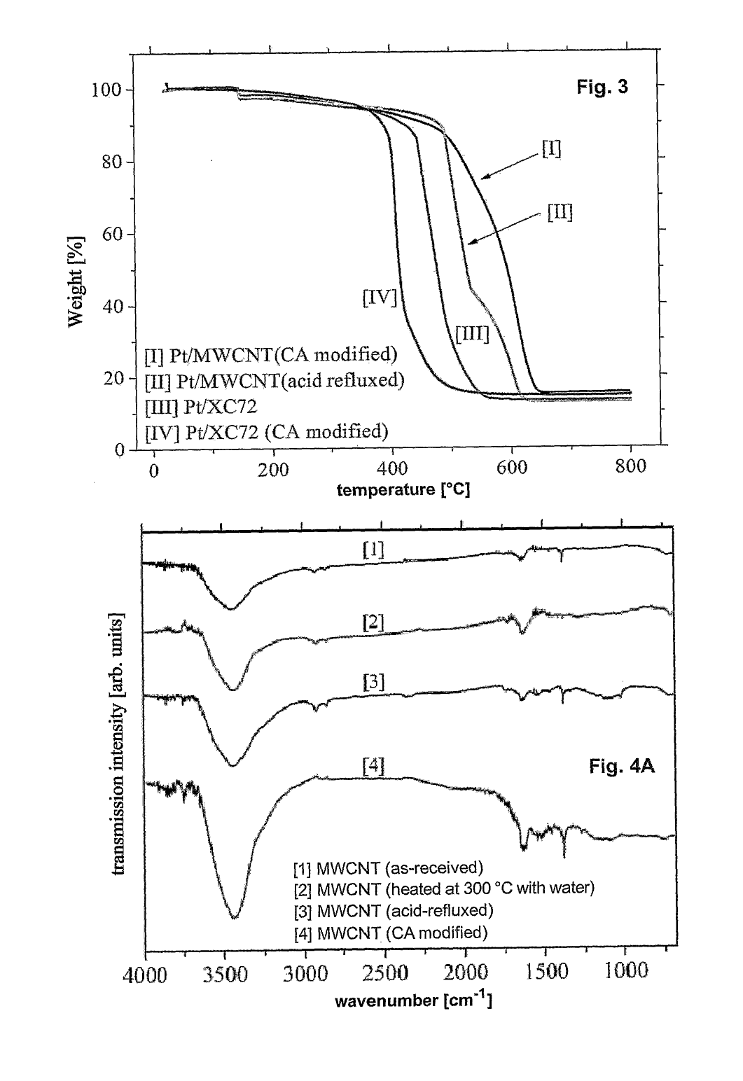

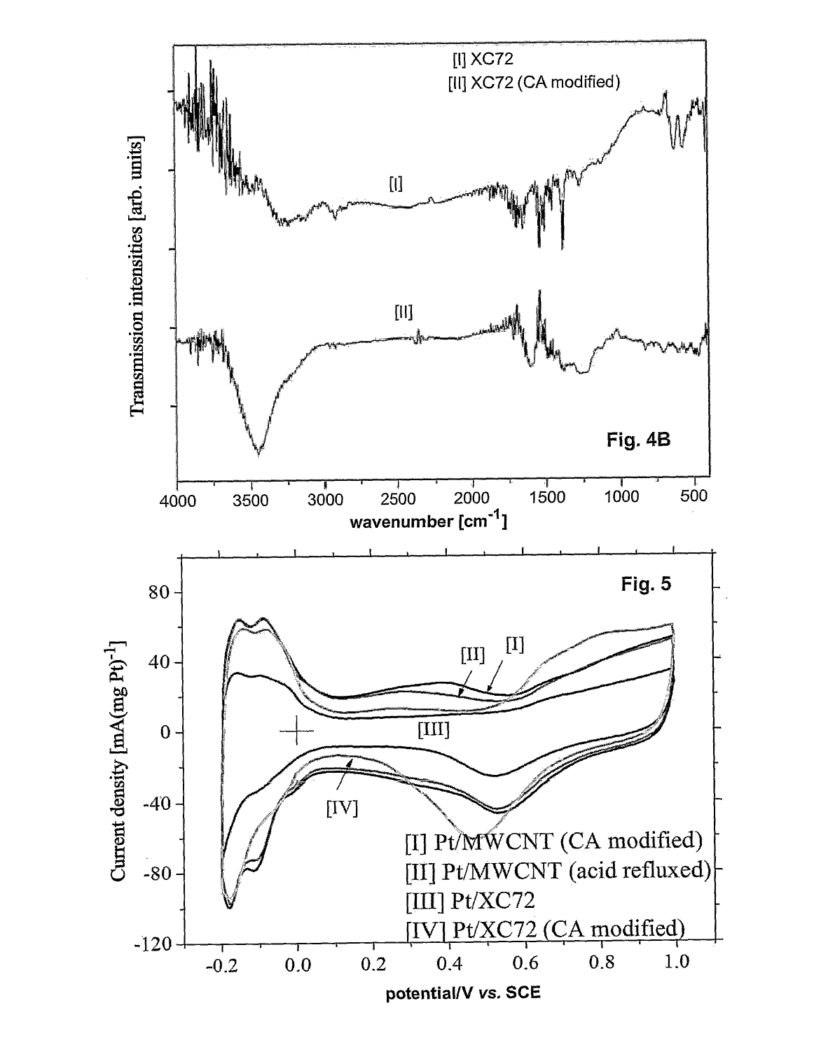

The FTIR spectra in FIG. 4 clearly show the existence of carbonyl and carboxyl groups within the wavenumber range 1300-1700 cm⁻¹ and the hydroxyl bands at a wavenumber range of 3300-3500 cm⁻¹ on all the carbon materials. They are particularly strong on multi-walled carbon nanotubes treated by the method of the invention using citric acid (Spectrum 4, FIG. 4A) and weak on as-purchased multi-walled carbon nanotubes (Spectrum 1, FIG. 4A). For the citric acid-treated multi-walled carbon nanotubes the bands at 1630 and 1380 cm⁻¹ may be due to the asymmetric and symmetric HCOO⁻ stretching. These assignments are in accordance to the fact that CH₂COOH is part of citric acid molecule. A similar experiment was also carried out applying the same heat treatment on the multi-walled carbon nanotubes but with no addition of citric acid. No IR band at 1380 cm⁻¹ is observable in spectrum 2 of FIG. 4A, which confirms that the functional groups were largely caused by citric acid and not by merely heating in water. As shown in FIG. 4B, the intensification and broadening of IR absorption bands of XC72 carbon blacks after citric acid modification clearly shows that there are more functional groups attached to the carbon blacks after the treatment.

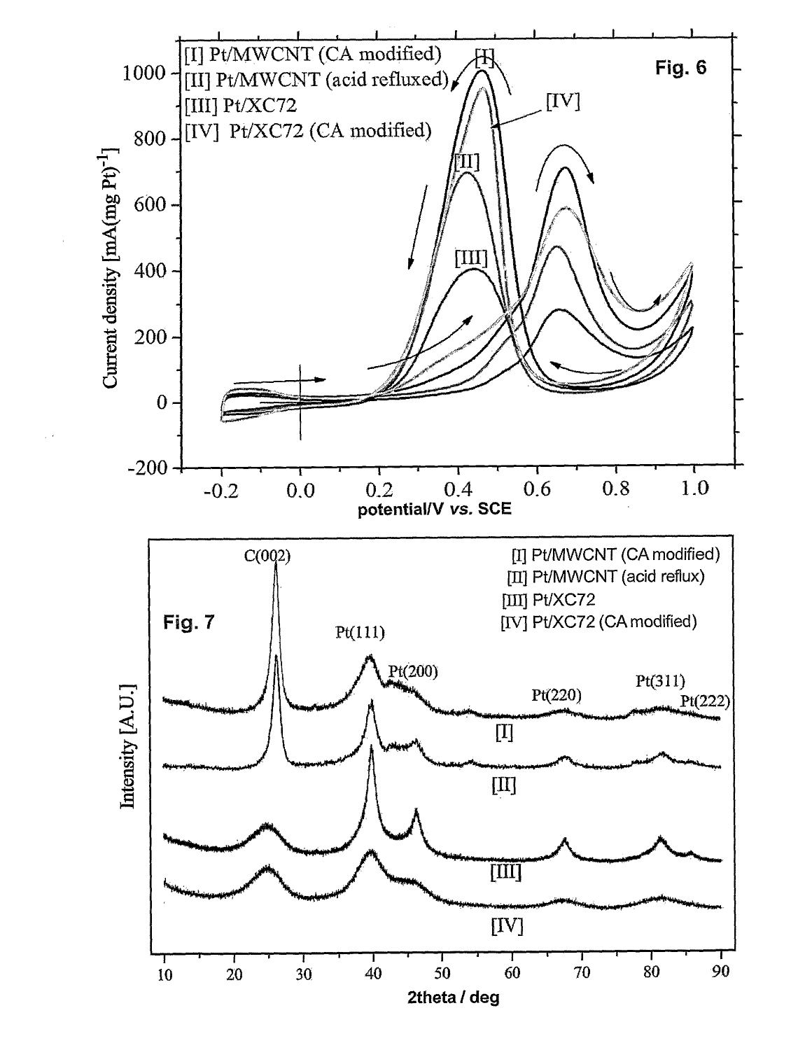

The cyclic voltammetry (CV) curves in FIG. 5 and FIG. 6 were obtained on the Pt catalysts on four different carbon supports in the potential range of −0.2 V to 1.0 V (vs. a reference saturated calomel electrode). From FIG. 5, it can be seen that Pt/multi-walled carbon nanotubes (both modified using citric acid and acid refluxed) and Pt/XC72 (modified using citric acid) produce much higher current density in the hydrogen adsorption/desorption region (−0.2 V-0.16 V) than Pt/XC 72. The capacitive current in the CV curves of the Pt/multi-walled carbon nanotubes catalyst (both citric acid modified and acid refluxed) is also higher than commercial carbon black due to the high specific capacitance of carbon nanotubes (Chen, J. H., et al., Carbon (2002) 40, 8, 1193-1197; Xing, Y., et al., Langmuir (2005) 21, 9, 4185-4190). The electrochemical active surface area of all the three Pt/C catalysts can be estimated from the hydrogen adsorption/desorption peaks of the cyclic voltammograms in FIG. 5 and FIG. 6. Assuming a hydrogen monolayer adsorption charge of Q_{H}⁰=210 μC/cm², (Le Gratiet, B., et al., J. Catal. (1996) 164, 1, 36-43) then the electrochemical active surface area (EAS) is given by S_{ec}=Q_{H}/Q_{H}⁰, where Q_{H} is the average specific charge derived from the hydrogen adsorption/desorption peaks area in the CV curve (Lordi, V., et al., Chem. Mater. (2001) 13, 3, 733-737). The EAS of the four catalysts are 73.8, 70.7, 43.5 and 76.02 m²/g for Pt/multi-walled carbon nanotubes (citric acid modified), Pt/multi-walled carbon nanotubes (acid refluxed), Pt/XC72 and Pt/XC72 (citric acid modified) respectively, as listed in Table 1 below. Table 1 shows the electrochemical active surface area calculated for the four indicated catalysts (CA modified=modified using citric acid, MWCNT=multi-walled carbon nanotubes). Compared to the high electrochemical surface area of the functionalized multi-walled carbon nanotubes and XC72, the electrochemical active surface (EAS) of Pt/XC72 is rather low, due to the large average particle size and poor dispersion of the Pt nanoparticles (see Table 1).

TABLE 1

| Type of Catalysts | S_{ec} (m²/g) |
|---|---|
| Pt/MWCNT (CA modified) | 73.82 |
| Pt/MWCNT (acid refluxed) | 70.71 |
| Pt/XC72 | 43.45 |
| Pt/XC72 (CA modified) | 76.02 |

The geometrical active surface area of the catalysts can be obtained from S_{geo} 6/(ρ×d), where ρ is the density of Pt and d is the average diameter of the particles [Thompsett, D., in Fuel Technology Handbook, CRC Press, 2003, chapter 6.2]. The geometrical active surface areas of the catalysts are 97.43, 90.32 and 87.81 m²/g for Pt/CNT (citric acid modified), Pt/CNT (acid refluxed) and Pt/XC-72 respectively. Comparing the EAS areas of the catalysts to their respective geometrical active surface areas, both Pt/CNT (citric acid modified) and Pt/CNT (acid refluxed) show higher percentage of electrochemically active Pt particles, while for Pt/XC-72 the EAS area is only 50% of its total geometrical active surface area (Table 1). This might be due to the poisoning of the Pt sites by sulfur contained in the carbon black, and thus a reduced in the electrochemically active Pt sites (Swider, K. E, & Rolison D. R., J. Electrochem. Soc. (1996) 143, 3, 813-81936; Tang, H., et al., Mater. Chem. Phys. (2005) 92, 2-3, 548-553). On the contrary, carbon nanotubes are produced from sulfur-free processes and thus has no or negligibly low sulfur content.

The cyclic voltammograms of methanol oxidation on the catalysts under the potential range of −0.2 V to 1.0 V (saturated calomel electrode (SCE)) are shown in FIG. 6, in which two peaks of methanol oxidation can be observed, i.e. Eₚ₁ (0.65-0.67 V) in the forward scan and Eₚ₂ (0.44-0.46 V) in the reversed scan. The shape of the CV and the peak potentials are accordant with other works (Swider, et al., 2005, supra). The specific current generated by Pt/multi-walled carbon nanotubes (modified using citric acid) at Eₚ₁ which corresponds to the methanol electroxidation is 0.64 A/(mgPt), which is about 2.5 times as large as that of Pt/XC72 and 1.5 times of Pt/multi-walled carbon nanotubes (acid refluxed). The high activity of Pt/multi-walled carbon nanotubes (modified using citric acid) may be attributed to several factors. According to the ab initio density-functional-theory calculations by Britto et al. (Adv. Mater. (1999) 11, 2, 154-157) carbon nanotube electrodes can improve charge transfer processes due to the unique structure of carbon nanotubes. The functional groups attached to the walls of carbon nanotubes are found to further enhance the conductivity of carbon nanotubes (Pan, H., et al., Phys. Rev. B (2004) 70, 24, 245425-1-245425-5). More importantly the functionalization of carbon nanotubes according to the method of the present invention introduced a lot of hydroxyl functional groups that might facilitate the removal of CO intermediate that adsorbed on Pt surface. Pt/XC72 (modified using citric acid) (curve IV in FIG. 6) also showed much higher oxidation peaks compared to the Pt catalyst supported on the as-purchased XC72 carbon blacks.

The electro-oxidation of methanol and the oxidation of CO by Pt catalyst can be summarized as follows (Kabbabi, A., et al., J. Electroanal. Chem. (1998) 444, 1, 41-53):

Pt+CH₃OH→Pt—CO_{ads}+4H⁺+4e⁻ (1)

Pt+H₂O→Pt—OH_{ads}+H⁺+e⁻ (2)

Pt—CO_{ads}+Pt—OH_{ads}→CO₂+H⁺+e⁻ (3)

On a pure Pt electrode, the rate of stripping the CO intermediate from Pt site is low since the adsorption of OH intermediate on Pt is difficult (Perez, A., et al., J. Phys. Chem. B (2005) 109, 49, 23571-23578). The presence of high concentration of hydroxyl groups on the carbon nanotubes can facilitate the removal of CO, preventing rapid decrease in the rate of dehydrogenation and thus the carbon nanotubes supported catalysts produced higher oxidation current compare to carbon black supported catalysts. As shown in FIG. 6, the current peaks in the reversed scan which are related to the oxidation of CO intermediates (Lee, J., et al., Electrochimica Acta (2002) 47, 13-14, 2297-2301) are higher for Pt/multi-walled carbon nanotubes (modified using citric acid) and Pt/XC72 (modified using citric acid) than the others.

X-ray diffraction (XRD) patterns of the catalysts are shown in FIG. 7. It can be seen that the crystal structure of Pt in the Pt/multi-walled carbon nanotube nanocomposites and Pt/XC72 is face-centered cubic (fcc), which is confirmed by the presence of diffraction peaks at 39.6°, 46.3°, 67.4°, 81.4°, and 85.4° (Tian, Z. Q., et al., J. Phys. Chem. B (2006) 110, 5343-53503). These peaks are assigned to Pt(111), Pt(200), Pt(220), Pt(311), and Pt(222), respectively. The average particle size of Pt nanoparticles was 2.5 nm (Pt/multi-walled carbon nanotubes, citric acid modified), 3.9 nm (Pt/multi-walled carbon nanotubes, acid refluxed), 6.4 nm (Pt/XC72, as purchased), and 2.4 nm (Pt/XC72, modified using citric acid), respectively, as determined by Sherrer's formula through line broadening of the Pt(111) peak (FIG. 7). The average particle sizes obtained from the XRD patterns are similar to the average particle sizes obtained from the TEM images.

In the present embodiment, citric acid was used to create functional groups on carbon nanotubes for the subsequent uniform dispersion of Pt or Au nanoparticles. The surface modification of multi-walled carbon nanotubes by a carboxylic acid has several advantages over the conventional reflux treatment process. It is done simply by heating the mixture of carboxylic acid and carbon material at about 300° C. for ½ h, while it usually takes 4 to 48 h in the reflux treatment process. As the thermal decomposition temperature of citric acid is 175° C., it is unlikely to have non-reacted acid in the carboxylic acid-treated multi-walled carbon nanotubes, thus washing and filtrating processes to remove the acid are unnecessary. Hence carboxylic acid modification of a carbon material is a simple and fast process. When multi-walled carbon nanotubes functionalized by citric acid treatment were employed as the support for Pt deposition, higher Pt loading, smaller particle-size and higher catalyst activity for fuel cell processes were measured as compared to those on acid-refluxed multi-walled carbon nanotubes under identical experimental conditions. Commercially available carbon blacks (XC72) were also functionalized and tested under similar conditions. Catalyst prepared using citric acid modified XC72 carbon blacks were also compared with catalyst prepared using as-purchased XC72 carbon blacks for their electrochemical performance.

In summary it has been shown that the present invention provides a simple and efficient method for functionalizing carbon materials and for forming highly dispersed metal nanoparticles on carbon materials. Citric acid modified multi-walled carbon nanotubes are shown by FTIR to have more functional groups on the surface of carbon nanotubes when compared to acid refluxed multi-walled carbon nanotubes. A higher degree of functionalization has previously been shown to improve solubility of carbon nanotubes (Dyke, C. A., & Tour, J. M., Chem. Eur. J. (2004) 10, 812-817). Furthermore, from the CV in 0.5 M sulfuric acid and methanol oxidation, Pt nanoparticles supported on citric acid modified multi-walled carbon nanotubes have higher activity than Pt supported on acid refluxed multi-walled carbon nanotubes. The current density produced by Pt catalyst supported on citric acid modified multi-walled carbon nanotubes and XC72 carbon blacks are larger than the Pt catalysts supported on acid refluxed multi-walled carbon nanotubes and as-purchased XC72 carbon blacks. This is a result of the higher density of functional groups produced by the method of the invention. A high density of functional groups can facilitate the dispersion of Pt catalysts and may enhance the removal of CO intermediates during the electrochemical processes.

### Example 1

### Citric Acid Treatment of Multi-Walled Carbon Nanotubes

The present example illustrates an embodiment of functionalizing carbon nanotubes as a model carbon material. Pt nanoparticles are then formed on the carbon nanotubes, thereby obtaining a catalyst for a fuel cell.

The average length of carbon nanotubes used in the current example was ˜2 μm. The carbon nanotubes were suspended in deionized water (DI water). All dielectrophoresis experiments were done under standard room temperature conditions.

In a typical experiment, 100 mg of multi-walled carbon nanotubes (purchased from Shenzhen Nanotech Co. Ltd. with diameters between 20 to 40 nm), 100 mg of citric acid monohydrate (Fluka 99.5%) and 10 ml of distilled water were mixed with the help of ultrasonic vibration (Elma, 100 W and 35 kHz) for 15 min, and then let dried to form a paste. After heated at 300° C. for 30 min, the citric acid treated multi-walled carbon nanotubes were ready for Pt deposition. The same procedure was repeated for XC72 carbon blacks.

### Example 2

### Deposition of Platinum Nanoparticles on Multi-Walled Carbon Nanotubes

40 mg of the above functionalized multi-walled carbon nanotubes were dispersed in 50 ml of ethylene glycol (Sigma Aldrich 99+%) by ultrasonic vibration and mixed with 1.0 mL of 0.04M H₂PtCl₆.6H₂O (Fluka) aqueous solution in a Teflon vessel. 0.5 mL of 0.8 M NaOH was added drop wise into the mixture and stirred vigorously. The mole ratio of NaOH/Pt was >8 to induce small and uniform Pt particles formation (Yu, W., et al., Langmuir (1999) 15, 1, 6-9). The Teflon vessel with the mixture was placed in the Milestone MicroSYNTH programmable microwave system (1000 W, 2.45 GHz), heated to 160° C. within 2 min, and maintained at the same temperature for 2 min for the reduction of the platinum precursor. The resulting suspension of Pt-deposited carbon nanotubes were centrifuged, washed with acetone to remove the organic solvent, and dried at 80° C. overnight in a vacuum oven.

To compare the carbon nanotubes modified using citric acid with conventional carbon supports, depositions of Pt nanoparticles were also conducted on acid-refluxed multi-walled carbon nanotubes, citric acid modified XC72 and as-purchased carbon blacks (XC72, Cabot Corp.) respectively under the same conditions described above. The acid-refluxed multi-walled carbon nanotubes were prepared by the refluxing of multi-walled carbon nanotubes with a concentrated H₂SO₄—HNO₃ acid (3:1 v/v) for 5 h, which were then filtered, washed and dried in a vacuum oven.

### Example 3

### Catalyst Characterization

The Pt particle size distribution was examined using TEM (JEOL JEM2010F) operating at 200 kV. A total of 400 Pt nanoparticles were counted in each sample to ensure statistically representative of the particle distribution.

The platinum loading of the catalyst was determined using a thermogravimetry analyzer (TGA) (Setaram TGA equipment). Several milligrams of the Pt/carbon samples were heated to 800° C. in the flow of purified oxygen.

The infrared transmission spectra were measured with a Perkin-Elmer 2000 Fourier-Transform Infrared Spectrometer (FTIR) in the range of 400 to 4000 cm⁻¹.

X-ray diffraction (XRD) measurements, were carried out using a Bruker D8 Advance X-Ray Diffractometer, scanned from 20=10° to 90°. The average crystallite size of Pt particles was estimated from the diffraction peak of Pt(111) using the Debye-Scherrer equation (Antolini, E., & Cardellini, F., J. Alloys Comp. (2001) 315, 118):

d=0.9λ_{kα1}/B cos θₘₐₓ (1)

in which d is the average size of the Pt particle, λ_{kα1} is the X-ray wavelength (Cu Kα λ_{kα1}=1.5418 Å), θₘₐₓ is the maximum angle of the (111) peak, and B is the full-width at half-maximum in radians.

### Example 4

### Electrochemical Measurement

Cyclic voltammetry (CV) measurements were performed using Solartron SI1280B, a combined electrochemical interface and frequency response analyzer, at room temperature with a scan rate of 50 mV/s. The working electrode was fabricated by casting a Nafion-impregnated catalyst ink onto a 3 mm diameter glassy carbon electrode. Typically 8 mg of the Pt/C catalyst dispersed in 0.5 mL of ethanol aqueous solution (1:1 v/v) was sonicated for 15 min and 60 μL of 5 wt % Nafion solution was added as polymer binder (Li, G., & Pickup, P. G.; J. Electrochem. Soc. (2003) 150, 11, C745-C752). 3.4 μL of this catalyst ink was dropped onto the glassy carbon electrode. The catalyst cast electrode was placed in a vacuum oven until the catalyst was totally dry. For the CV measurement the catalyst cast working electrode was immersed in 0.5 M H₂SO₄ with or without 1 M CH₃OH which was deaerated with high purity nitrogen gas for electrochemical measurement. A Pt foil and a saturated calomel electrode (SCE) were used as counter electrode and reference electrode respectively.

### Example 5

### Citric Acid Treatment of XC72 Carbon Blacks

In a typical experiment, 100 mg of XC72 carbon blacks (Cabot Corporation), 100 mg of citric acid monohydrate (Fluka 99.5%) and 10 ml or more of distilled water were mixed with the help of ultrasonic vibration (Elma, 100 W and 35 kHz) for 15 min, and then let dried to form a paste. After being heated at 300° C. for 30 min, the citric acid treated XC72 carbon blacks were ready for Pt deposition.

The listing or discussion of a previously published document in this specification should not necessarily be taken as an acknowledgement that the document is part of the state of the art or is common general knowledge. All documents listed are hereby incorporated herein by reference in their entirety for all purposes.

The invention illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. A method of functionalizing a carbon material, the method comprising:
contacting a carbon material with a carboxylic acid, thereby forming a mixture;
heating the mixture for a suitable period of time at a temperature below the thermal decomposition temperature of the carbon material, thereby forming a functionalized carbon material.

2. The method of claim 1, wherein the carbon material is crystalline carbon.

3. The method of claim 1 or 2, wherein the carbon material comprises one of carbon black, a carbon nanofilament, a 3D carbon sieve, activated carbon, graphite and a carbide-derived carbon material.

4. The method of claim 3, wherein the carbon nanofilament is one of a carbon nanotube, a carbon nanohorn and a carbon nanowire.

5. The method of any one of claims 1-4, wherein the carbon material is contacted with a solution of the carboxylic acid.

6. The method of any one of claims 1-5, wherein the carbon material is contacted with the carboxylic acid by adding a solution of the carboxylic acid to the carbon material.

7. The method of any one of claims 1-6, wherein contacting the carbon material with the carboxylic acid comprises forming a suspension and/or a solution of the carboxylic acid and the carbon material in a solvent.

8. The method of any one of claims 1-7, wherein the carboxylic acid is one of a hydroxy carboxylic acid, a dicarboxylic acid, an amino acid and any combination thereof.

9. The method of any one of claims 1-8, wherein the carboxylic acid is selected from the group consisting of oxalic acid, ascorbic acid, citric acid, glycolic acid, tartaric acid, malic acid, maleic acid, adipic acid, lactic acid, salicylic acid and any mixture thereof.

10. The method of claim 9, wherein the amino acid is selected from the group consisting of Glutamine, Lysine, Histidine, Serine, Threonine, Tyrosine, Cystine, Arginine, Proline, Glutamic acid, Aspartic acid, Asparagine and Glutamine.

11. The method of any one of claims 5-10, wherein the solution of the carboxylic acid is an aqueous solution.

12. The method of any of claims 1-11, further comprising intermixing the mixture of the carbon material and the carboxylic acid.

13. The method of any one of claims 5-12, wherein contacting the carbon material with the solution of a carboxylic acid comprises applying ultrasound.

14. The method of any of claims 1-13, further comprising:
drying the mixture of the carbon material and the carboxylic acid.

15. The method of claim 14, wherein drying the mixture comprises heating the mixture at a temperature below the thermal decomposition temperature of the carboxylic acid.

16. The method of claim 14 or 15, wherein the dried mixture is heated for a suitable period of time at a temperature below the thermal decomposition temperature of the carbon material.

17. The method of any one of claims 14-16, wherein by drying a paste is formed.

18. The method of any one of claims 1-17, wherein heating the mixture for a suitable period of time at a temperature below the thermal decomposition temperature of the carbon material is carried out at a temperature above the thermal decomposition temperature of the carboxylic acid.

19. The method of any one of claims 1-18, wherein heating the mixture of the carbon material and the carboxylic acid at a temperature below the thermal decomposition temperature of the carbon material is carried out for about 3 or 2 hours or less.

20. The method of claim 19, wherein heating the mixture of the carbon material and the carboxylic acid at a temperature below the thermal decomposition temperature of the carbon material is carried out for about 30 min to about 60 min.

21. The method of any one of claims 1-20, wherein heating the mixture of the carbon material and the carboxylic acid at a temperature below the thermal decomposition temperature of the carbon material is carried out in a furnace.

22. The method of any one of claims 1-21, wherein the functionalized carbon material comprises a functional group selected from the group consisting of —COOH, —CHO, —CO—, —OSO₃H and —OH.

23. The method of any one of claims 1-22, further comprising comminuting the functionalized carbon material.

24. A method of immobilizing matter on a carbon material, the method comprising:
contacting a carbon material with a carboxylic acid, thereby forming a mixture;
heating the mixture for a suitable period of time at a temperature below the thermal decomposition temperature of the carbon material, thereby forming a functionalized carbon material; and
contacting the functionalized carbon material with a compound capable of forming a covalent bond and/or an ionic bond with the functional groups on the functionalized carbon material.

25. The method of any of claim 24, further comprising:
drying the mixture of the carbon material and the carboxylic acid.

26. The method of claim 24 or 25, wherein the carbon material is contacted with a solution of a carboxylic acid.

27. The method of any one of claims 24-26, wherein the compound capable of forming a covalent bond and/or an ionic bond with the functional groups on the functionalized carbon material is provided in a solvent.

28. The method of any one of claims 24-27, wherein the compound capable of forming a covalent bond and/or an ionic bond with the functional groups on the functionalized carbon material is a metal compound or a metalloid compound.

29. The method of claim 28, wherein the method is a method of forming particles on a carbon material, the particles being at least one of a metal, a metal oxide, a metalloid and a metalloid oxide.

30. The method of claim 29, wherein the metal is a noble metal.

31. The method of claim 30, wherein the noble metal is selected from the group consisting of rhodium, rhenium, ruthenium, palladium, platinum, silver, gold and any combination thereof.

32. The method of any one of claims 24-27, wherein the compound capable of forming a covalent bond and/or an ionic bond with the functional groups on the functionalized carbon material is comprised in a particle.

33. The method of any one of claims 24-32, further comprising comminuting the functionalized carbon material.

34. The method of claim 33, wherein the functionalized carbon material is comminuted before contacting the same with a metal compound capable of forming a covalent bond and/or an ionic bond with the functional groups on the functionalized carbon material.

35. The use of a carbon material with particles obtained by the methods of any one of claims 29 to 31 in catalysis.

36. The use of claim 35, wherein the catalysis is in oxidation and/or reduction in a fuel cell.
